(19) United States
(12) Patent Application Publication
(10) Pub. No.: US 2024/0041856 A1
(43) Pub. Date: Feb. 8, 2024

(54) LIQUID APIXABAN FORMULATION IN SMALL DOSE VOLUME

(71) Applicant: DAFECHEM LIMITED, 'Egomi (CY)

(72) Inventor: Matthaios VIDALIS, Is-Swatar (MT)

(21) Appl. No.: 18/256,786

(22) PCT Filed: Dec. 13, 2021
(86) PCT No.: PCT/EP2021/085411

§ 371 Date: Jun. 9, 2023

(30) Foreign Application Priority Data
Dec. 13, 2020 (EP) EP20213637

(51) Int. Cl. A61K 31/4545; A61K 9/00; A61K 9/08; A61K 47/10; A61K 47/46

(52) U.S. Cl. CPC A61K 31/4545; A61K 9/0053; A61K 9/08; A61K 47/10; A61K 47/46

(57) ABSTRACT

A pharmaceutical liquid formulation for oral delivery of apixaban comprising i) apixaban in high concentrations, ii) more than 35% (w/v) propylene glycol based on the total formulation and ii) optionally other pharmaceutical acceptable excipients, wherein the formulation is suitable for small dose volume administration. The invention relates to a liquid formulation, which can be useful as pharmaceutical formulations, for oral delivery of apixaban with a small dose volume.

### FIELD OF THE INVENTION

This invention relates to a liquid formulation, which can be useful as pharmaceutical formulations, for oral delivery of apixaban with a small dose volume.

### BACKGROUND OF THE INVENTION

Apixaban is a highly selective inhibitor of factor Xa. It is categorized as an anti-coagulant medication, a blood thinner. It is used to treat and prevent blood clots following hip or knee replacement and to prevent stroke and systemic embolism in people with nonvalvular atrial fibrillation.

Apixaban is chemically described as 1-(4-methoxyphenyl)-7-oxo-6-[4-(2-oxopiperidin-1-yl)phenyl]-4,5,6,7-tetrahydro-1H-pyrazolo[3,4c]pyridine-3-carboxamide (IUPAC name) and is marketed under the name Eliquis®. Apixaban was approved by the FDA on Dec. 28, 2012, as tablets for oral use.

Formulations in tablet form comprising apixaban for oral administration are known. Liquid formulations comprising apixaban and a vehicle are described in WO2014052678A1. WO2014052678A1 discloses an oral liquid formulation comprising apixaban and a vehicle comprising water which results into relatively high dose volumes (˜25 mL). Especially patients undergoing hip surgery and geriatric patients who are usually unwilling and/or unable to swallow high dose volumes, high dose volume cause discomfort. Discomfort while swallowing, especially for bedridden patients are the most important parameters governing patient compliance. Liquid medicaments intended for the oral administration are advantageous to be administrated in small dose volume of less than 10 mL, especially for geriatric, pediatric patients and patients undergoing hip surgery who are bedridden. Small dose volume of liquid medicaments are also advantageous for patients who usually receive their medication through feeding tubes. Preferably the patient is a human patient.

Hence, it was an object of the present invention to provide a small dose volume apixaban oral formulation which minimizes patient discomfort. Furthermore, the pharmaceutical liquid formulation should show an appropriate storage stability and/or should show a low tendency for degradation of apixaban.

### BRIEF SUMMARY OF THE INVENTION

It is therefore an aim of the present invention to provide an optimized apixaban pharmaceutical formulation suitable for oral administration which overcomes the above-mentioned problems.

It is a further aim of the present invention to provide an improved method of manufacturing an apixaban pharmaceutical formulation suitable for oral administration.

It is a yet further aim of the present invention to provide a kit of use of an improved apixaban pharmaceutical formulation for oral administration.

It is a yet further aim of the present invention to provide a method of use of an improved apixaban pharmaceutical formulation for oral administration which can be adjustable to the prescribed dosing scheme and can be administrated in multiple dosing times.

In some embodiments, the pharmaceutical liquid formulation has a dose volume of equal to or less than 10 mL, preferably equal to or less than 5 mL, more preferably equal to or less than 4 mL, more preferably equal to or less than 3 mL, even more preferably equal to or less than 2 mL, preferably equal to or less than 1 mL. In some embodiments, the pharmaceutical liquid formulation comprises more than 35% (w/v) propylene glycol based on the total formulation. In some embodiments the pharmaceutical liquid formulation comprises more than 40% (w/v) propylene glycol based on the total formulation. Preferably, the pharmaceutical liquid formulation comprises more than 45% (w/v) propylene glycol based on the total formulation. Even more preferably, the pharmaceutical liquid formulation comprises more than 50% (w/v) propylene glycol based on the total formulation.

In some embodiments, the pharmaceutical liquid formulation further comprises an antioxidant, preferably butylated hydroxytoluene (BHT), butylated hydroxyanisole (BHA), propyl gallate, phospholipids and/or mixtures thereof.

In some embodiments the pharmaceutical kit is comprising a container comprising the pharmaceutical liquid formulation according to any one of the embodiments, (ii) optionally, a calibrated device, and optionally (iii) instructions for administration.

Preferably the container is of glass and/or plastic material and more preferably is an amber glass bottle. In some embodiments the container is a multi-use container. In some embodiments the container is a single-use container. Preferably, in some embodiments the calibrated device is selected from an oral syringe, a dropper, or a spoon.

Embodiment 1: A pharmaceutical liquid formulation for oral delivery of apixaban comprising apixaban at concentrations within the range of 0.25 mg/mL to 10 mg/mL and more than 35% (w/v) propylene glycol based on the total formulation wherein the dose volume of the liquid formulation is equal to or less than 10 mL.

Embodiment 2: A pharmaceutical liquid formulation for oral delivery of apixaban comprising apixaban at concentrations within the range of 1.25 mg/mL to 2.5 mg/mL and more than 35% (w/v) propylene glycol based on the total formulation wherein the dose volume of the liquid formulation is equal to or less than 5 mL.

Embodiment 3: A pharmaceutical liquid formulation for oral delivery of apixaban of embodiments 1, 2 wherein the formulation is comprising more than 45% (w/v) v/v propylene glycol based on the total formulation.

Embodiment 4: A pharmaceutical liquid formulation for oral delivery of apixaban of embodiment 3, wherein the formulation is a solution.

Embodiment 5: A pharmaceutical liquid formulation for oral delivery of apixaban of embodiment 4, wherein the formulation is essentially non-aqueous.

Embodiment 6: A pharmaceutical liquid formulation for oral delivery of apixaban according to any of the embodiments wherein the formulation further comprises antioxidants.

Embodiment 7: A pharmaceutical liquid formulation for oral delivery of apixaban according to embodiment 6, wherein the formulation further comprises antioxidants selected from butylated hydroxytoluene (BHT), butylated hydroxyanisole (BHA), propyl gallate, phospholipids and/or mixtures thereof.

Embodiment 8: A pharmaceutical liquid formulation for oral delivery of apixaban according to embodiment 1 and embodiment 2 wherein the formulation further comprises co-solvents selected from the group of polyethylene glycol, ethanol, water, glycerol, sorbitol, sucrose, mannitol, maltitol, fructose, glucose and/or mixtures thereof.

Embodiment 9: A pharmaceutical liquid formulation for oral delivery of apixaban according to any of the embodiments, wherein the formulation comprises one or more sweetening agents and/or flavoring agents.

Embodiment 10: A pharmaceutical liquid formulation for oral delivery of apixaban according to any of the embodiments, wherein the formulation further comprises a surfactant.

Embodiment 11: A method for preparing a pharmaceutical liquid formulation for oral delivery of apixaban according to any proceeding claims comprising
i. mixing apixaban and optionally pharmaceutically acceptable excipients in
   propylene glycol and optionally in co-solvents
ii. apply heat temperature up to 90° C.
iii. maintaining temperature while mixing apixaban until apixaban dissolves
iv. optionally, bringing to volume with propylene glycol and/or co-solvent and optionally
   sweetening agents and/or flavoring agents.
v. optionally, packing in a container

Embodiment 12: A method for preparing a pharmaceutical liquid formulation for oral delivery of apixaban according to any of the embodiments, comprising
i. mixing apixaban and optionally pharmaceutically acceptable excipients in at
   least 35% (w/v) propylene glycol and optionally in co-solvents
ii. apply heat temperature up to 90° C.
iii. maintaining temperature while mixing apixaban until apixaban dissolves
iv. if required, bringing to volume with propylene glycol and/or co-solvent and
   optionally sweetening agents and/or flavoring agents.
v. optionally, packing in a container

Embodiment 13: A pharmaceutical kit comprising: (i) a container comprising the pharmaceutical liquid formulation according to any of the embodiments, (ii) optionally a calibrated device, and optionally (iii) instructions for administration.

Embodiment 14: A pharmaceutical kit according to embodiment 13, wherein the container comprising the pharmaceutical liquid formulation is glass and/or plastic material.

Embodiment 15: A pharmaceutical kit according to embodiments 14, wherein the calibrated device is selected from an oral syringe, a dropper, or a spoon.

Embodiment 16: A pharmaceutical liquid formulation for oral delivery of apixaban according to any of the embodiments for use as a therapy of blood clots following hip or knee replacement, stroke and systemic embolism in people with nonvalvular atrial fibrillation.

Embodiment 17: A pharmaceutical liquid formulation for oral delivery of apixaban according to the proceeding claims for use as a therapy of blood clots following hip or knee replacement, stroke and systemic embolism in people with nonvalvular atrial fibrillation wherein the dose volume is administered at least partially and at least once per day.

### DETAILED DESCRIPTION OF THE INVENTION

The present invention relates to a pharmaceutical liquid formulation for oral delivery of apixaban comprising apixaban at concentrations within the range of mg/mL to 10 mg/mL, wherein the dose volume is equal to or less than 10 mL.

Surprisingly, a pharmaceutical liquid formulation for oral delivery of apixaban comprising apixaban at concentrations within the range of 0.25 mg/mL to 10 mg/mL and more than 35% (w/v) propylene glycol based on the total formulation, dissolves apixaban in small vehicle volumes, resulting in a solution which remains stable and is free of precipitation/crystallization.

The pharmaceutical liquid formulation of the invention is an oral formulation. It can be administrated orally by conventional means such as swallowing or can be administered through feeding tubes. Feeding tubes can be inserted via a number of routes: via the nasopharynx, for example nasogastric (NG) or nasojejunal (NJ), or via direct access to the GI tract through the skin, for example gastrostomy or jejunostomy tubes. The invention is suitable for use through nasogastric or other feeding tubes.

The pharmaceutical liquid formulation of the invention is adjustable to the prescribed dosing scheme and can be administrated flexibly according to the desired pharmaceutical dosing scheme. The small dose volume of the pharmaceutical liquid formulation of the invention can be multiplied or divided. Therefore, patient compliance is achieved and swallowing is facilitated. It can be administrated in multiple times during the day.

According to a first aspect, a pharmaceutical liquid formulation for oral delivery of apixaban comprising apixaban at concentrations within the range of 0.25 mg/mL to 10 mg/mL and more than 35% (w/v) propylene glycol based on the total formulation, wherein the dose volume of the liquid formulation is less than 10 mL. Preferably, said pharmaceutical liquid formulation is comprising at least 40% (w/v) propylene glycol based on the total formulation. Preferably, said pharmaceutical liquid formulation is comprising at least 45% (w/v) propylene glycol based on the total formulation. More preferably, said pharmaceutical liquid formulation is comprising at least 50% (w/w) propylene glycol based on the total formulation. Preferably, said pharmaceutical liquid formulation is comprising at least 55% (w/v) propylene glycol based on the total formulation.

A preferred embodiment of the invention is a pharmaceutical liquid formulation for oral delivery of apixaban comprising apixaban at concentrations within the range of 1.25 mg/mL to 2.5 mg/mL and more than 35% (w/v) propylene glycol based on the total formulation, wherein the dose volume of the liquid formulation is equal to or less than 10 mL. Preferably the does volume is equal to or less than 5 mL. Preferably, said pharmaceutical liquid formulation is comprising at least 40% (w/v) propylene glycol based on the total formulation. Preferably, said pharmaceutical liquid formulation is comprising at least 45% (w/v) propylene glycol based on the total formulation. Even more preferably, said pharmaceutical liquid formulation is comprising at least 50% (w/v) propylene glycol based on the total formulation.

According to a another aspect, the pharmaceutical liquid formulation for oral delivery of apixaban comprising apixaban at concentrations within the range of 0.25 mg/mL to mg/mL and more than 35% (w/v) propylene glycol based on the total for-mulation wherein the dose volume of the liquid formulation is less than 10 mL, wherein the formulation further comprises co-solvents such as polyethylene glycol PEGS (200-400), ethanol, water, sorbitol, sucrose, mannitol, maltitol, fructose, glucose, glycerol and mixtures thereof.

In particular, pharmaceutical liquid formulation for oral delivery of apixaban comprising apixaban at concentrations within the range of 0.25 mg/mL to 10 mg/mL and more than 35% (w/v) propylene glycol based on the total formulation, wherein the dose volume of the liquid formulation is equal to or less than 10 mL, is a solution. Preferably, the solution is essentially non-aqueous. The term “essentially non-aqueous” within the scope of the invention is to be understood that the formulation is substantially free of water.

According to another aspect, pharmaceutical liquid formulation for oral delivery of apixaban comprising apixaban at concentrations within the range of 0.25 mg/mL to 10 mg/mL and more than 35% (w/v) propylene glycol based on the total formulation wherein the dose volume of the liquid formulation is equal to or less than 10 mL, wherein the formulation optionally comprises an antioxidant. Preferably, the pharmaceutical liquid formulation may optionally comprise an antioxidant selected from butylated hydroxytoluene (BHT), butylated hydroxyanisole (BHA), propyl gallate, phospholipids and mixtures thereof.

According to another aspect, the pharmaceutical liquid formulation for oral delivery of apixaban comprising apixaban at concentrations within the range of 0.25 mg/mL to 10 mg/mL and more than 35% (w/v) propylene glycol based on the total formulation wherein the dose volume of the liquid formulation is equal to or less than 10 mL, wherein the formulation further comprises a surfactant. Preferably the surfactant is Sodium Lauryl Sulfate and/or Sodium Docusate.

According to another aspect, pharmaceutical liquid formulation for oral delivery of apixaban comprising apixaban at concentrations within the range of 0.25 mg/mL to 10 mg/mL and more than 35% (w/v) propylene glycol based on the total formulation wherein the dose volume of the liquid formulation is equal to or less than 10 mL, wherein the formulation further comprises sweetening agents and/or flavoring agents. Preferably, sweetening agents are sodium saccharin, neohesperidin dihydrochalcone, sorbitol, sucrose, mannitol, maltitol, fructose, and glucose whereas flavoring agents are selected from herbal origin such as peppermint, spearmint, and citrus fruits such as orange and lemon.

For apixaban solutions with concentrations of equal to 0.25 mg/mL, apixaban dissolves freely in the vehicle comprising at least 35% (w/v) propylene glycol based on the total formulation at room temperature.

However, for achieving solutions of apixaban with concentration of more than 0.5 mg/mL in a vehicle comprising at least 35% (w/v) propylene glycol based on the total formulation, heating within the range of 25-90° C. is applied so to achieve full and quick solubilization of apixaban. Apixaban in propylene glycol was found to be stable at temperature increase up to 90° C. to succeed appropriate solubilization.

Furthermore, the present inventors provide a process for preparing pharmaceutical liquid formulation for oral delivery of apixaban comprising apixaban at concentrations within the range of 0.25 mg/mL to 10 mg/mL and more than 35% (w/v) propylene glycol based on the total formulation wherein the dose volume of the liquid formulation is equal to or less than 10 mL, which said process comprises
i. mixing apixaban and optionally pharmaceutically acceptable excipients in
   propylene glycol and optionally in co-solvents
ii. apply heat temperature up to 90° C.
iii. maintaining temperature while mixing apixaban until apixaban dissolves
iv. optionally, bringing to volume with propylene glycol and/or co-solvent and
   optionally sweetening agents and/or flavoring agents.
v. optionally, packing in a container

Preferably, a process for preparing a pharmaceutical liquid formulation for oral delivery of apixaban comprising apixaban at concentrations within the range of 0.25 mg/mL to mg/mL and more than 35% (w/v) propylene glycol based on the total formulation, which said process comprises
i. mixing apixaban and optionally pharmaceutically acceptable excipients in at least
   35% (w/v) of propylene glycol and optionally in co-solvents
ii. apply heat temperature up to 90° C.
iii. maintaining temperature while mixing apixaban until apixaban dissolves
iv. optionally, bringing to volume with propylene glycol and/or co-solvent and
   optionally sweetening agents and/or flavoring agents.
v. optionally, packing in a container

More preferably, a process for preparing a process for preparing a pharmaceutical liquid formulation for oral delivery of apixaban comprising apixaban at concentrations within the range of 0.25 mg/mL to 10 mg/mL and more than 45% (w/v) propylene glycol based on the total formulation wherein the dose volume of the liquid for-mulation is equal to or less than 10 mL, which said process comprises, dissolving pharmaceutically acceptable excipients in at least 50% (w/v) of propylene glycol and optionally in co-solvents
i. mixing apixaban and pharmaceutically acceptable excipients in propylene glycol
   and optionally in co-solvents
ii. apply heat temperature up to 90° C.
iii. maintaining temperature while mixing apixaban until apixaban dissolves
iv. optionally, bringing to volume with propylene glycol and/or co-solvent and
   optionally sweetening agents and/or flavoring agents.
v. optionally, packing in a container

Preferably, the container is a glass and/or plastic container. More preferably the container is an amber glass container.

Another aspect of the invention is a pharmaceutical kit comprising: (i) a container comprising the pharmaceutical liquid formulation according to any embodiment, (ii) optionally, a calibrated device, and optionally (iii) instructions for administration. The calibrated device is preferably an oral syringe, a dropper, or a spoon. The calibrateddevice is calibrated to equal to or less than 10 mL. Preferably the calibrated device is calibrated to equal to or less than 5 mL. More preferably the calibrated device is calibrated to equal to or less than 2 mL. Even more preferably the calibrated device is calibrated to equal to or less than 1 mL.

In an embodiment of the invention the pharmaceutical kit is comprising a container which is a single-use container. The single-use container comprises the exact dose volume hence no calibrated device is packed. Optionally instructions for administration are comprised. In another embodiment of the invention the pharmaceutical kit is comprising a container which is a multi-use container and a calibrated device. The calibrated device is preferably an oral syringe, a dropper, or a spoon. Preferably the calibrated device is calibrated to 10 mL. More preferably the calibrated device is calibrated to 6 mL. Even more preferably the calibrated device is calibrated to 5 mL. Even more preferably the calibrated device is calibrated to 2 mL. Even more preferably the calibrated device is calibrated to 1 mL. Optionally instructions for administration are comprised.

### EXAMPLES

The pharmaceutical liquid formulations as described herein may be illustrated by the following examples which are not to be construed as limiting the scope of the invention. The Examples 1-6 contain 5 mg or 2.5 mg apixaban per dose corre-spondingly. The Examples 7-38 contain apixaban in various concentrations and are illustrative of apixaban's solubility.

### Example 1

### Manufacturing Process of Example 1a and Example 1b

Examples 1a and 1b were prepared according to the following steps:
i. Butylated hydroxyanisole is dissolved into PEG 200 and propylene glycol
ii. Heat the solution at the temperature of 40-45° C.
iii. Add apixaban and neohesperidin dihydrochalcone while maintaining the
   temperature until a clear solution forms
iv. Allow the solution to equilibrate at room temperature
v. Peppermint flavor is added and the volume of the solution is brought to 2 mL
   by adding the required amount of glycerol
vi. Pack the solution in amber glass bottles.

| | | Example 1a | | Example 1b | |
|---|---|---|---|---|---|
| | | Apixaban | | Apixaban | |
| | | concentration | | concentration | |
| | | 2.5 mg/mL | | 1.25 mg/mL | |
| No | Ingredients | mg/Dose | % | mg/Dose | % |
| 1 | Apixaban | 5.00 | 0.25 | 2.50 | 0.125 |
| 2 | Polyethylene Glycol | 200.00 | 10.00 | 200.00 | 10.00 |
| | 200 | | | | |
| 3 | Propylene Glycol | 1000.00 | 50.00 | 1000.00 | 50.00 |
| 4 | Neohesperidin Dihy- | 1.00 | 0.05 | 1.00 | 0.05 |
| | drochalcone | | | | |
| 5 | Butylated Hydroxy- | 0.20 | 0.01 | 0.20 | 0.01 |
| | anisole | | | | |
| 6 | Peppermint flavor | 3.00 | 0.15 | 3.00 | 0.15 |
| 7 | Glycerol anhydrous | qs to 2 | qs | qs to 2 | qs |
| | | mL | | mL | |
| | Total | 2.00 mL | | 2.00 mL | |

### Manufacturing Process of Example 2a and Example 2b

Examples 2a and 2b were prepared according to the following steps:
i. Butylated hydroxyanisole is dissolved into PEG 200 and about 60% propylene
   glycol
ii. Heat the solution at the temperature of 40-45° C.
iii. Add apixaban, and neohesperidin dihydrochalcone while maintaining the tem-perature
   until a clear solution forms
iv. Allow the solution to equilibrate at room temperature
v. Peppermint flavor is added and the volume of the solution is brought to 2 mL by adding
   the required amount of propylene glycol
vi. Pack the solution in amber glass bottles.

| | | Example 2a | | Example 2b | |
|---|---|---|---|---|---|
| | | Apixaban | | Apixaban | |
| | | concentration | | concentration | |
| | | 2.5 mg/mL | | 1.25 mg/mL | |
| No | Ingredients | mg/Dose | % | mg/Dose | % |
| 1 | Apixaban | 5.00 | 0.25 | 2.50 | 0.125 |
| 2 | Polyethylene Glycol | 200.00 | 10.00 | 200.00 | 10.00 |
| | 200 | | | | |
| 3 | Propylene Glycol | 1000.00 | 50.00 | 1000.00 | 50.00 |
| 4 | Neohesperidin Dihy- | 1.00 | 0.05 | 1.00 | 0.05 |
| | drochalcone | | | | |
| 5 | Butylated Hydroxy- | 0.20 | 0.01 | 0.20 | 0.01 |
| | anisole | | | | |
| 6 | Peppermint flavor | 3.00 | 0.15 | 3.00 | 0.15 |
| 7 | Propylene Glycol | qs to 2 | qs | qs to 2 | qs |
| | | mL | | mL | |
| | Total | 2.00 mL | | 2.00 mL | |

### Manufacturing Process of Example 3a and Example 3b

Examples 3a and 3b were prepared according to the following steps:
i. Sodium lauryl sulfate is added in about 60% propylene glycol
ii. Heat the mixture at the temperature of 40-45° C. until sodium lauryl sulfate is fully dissolved
iii. Add apixaban, PEG 200, propylene glycol and neohesperidin
   dihydrochalcone while maintaining the temperature, until a clear solution forms
iv. Allow the solution to equilibrate at room temperature
v. Peppermint flavor is added and the volume of the solution is brought to 2 mL
   by adding the required amount of glycerol
vi. Pack the solution in amber glass bottles.

| | | Example 3a | | Example 3b | |
|---|---|---|---|---|---|
| | | Apixaban | | Apixaban | |
| | | concentration | | concentration | |
| | | 2.5 mg/mL | | 1.25 mg/mL | |
| No | Ingredients | mg/Dose | % | mg/Dose | % |
| 1 | Apixaban | 5.00 | 0.25 | 2.50 | 0.125 |
| 2 | Polyethylene Glycol | 200.00 | 10.00 | 200.00 | 10.00 |
| | 200 | | | | |
| 3 | Propylene Glycol | 1000.00 | 50.00 | 1000.00 | 50.00 |
| 4 | Sodium Lauryl | 10.00 | 0.50 | 10.00 | 0.50 |
| | Sulfate | | | | |
| 5 | Neohesperidin Dihy- | 1.00 | 0.05 | 1.00 | 0.05 |
| | drochalcone | | | | |
| 6 | Peppermint flavor | 3.00 | 0.15 | 3.00 | 0.15 |
| 7 | Glycerol anhydrous | qs to 2 | qs | qs to 2 | qs |
| | | mL | | mL | |
| | Total | 2.00 mL | | 2.00 mL | |

### Manufacturing Process of Example 4a and Example 4b

Examples 4a and 4b were prepared according to the following steps:
i. Apixaban is added in propylene glycol and PEG-200
ii. Heat the mixture at the temperature of 40-45° C. until apixaban is fully
   dissolved
iii. Add neohesperidin dihydrochalcone while maintaining the temperature, until a
   clear solution forms
iv. Allow the solution to equilibrate at room temperature
v. Peppermint flavor is added and the volume of the solution is brought to 2 mL
   by adding the required amount of glycerol
vi. Pack the solution in amber glass bottles

| | | Example 4a | | Example 4b | |
|---|---|---|---|---|---|
| | | Apixaban | | Apixaban | |
| | | concentration | | concentration | |
| | | 2.5 mg/mL | | 1.25 mg/mL | |
| No | Ingredients | mg/Dose | % | mg/Dose | % |
| 1 | Apixaban | 5.00 | 0.25 | 2.50 | 0.125 |
| 2 | Polyethylene Glycol | 200.00 | 10.05 | 200.00 | 10.05 |
| | 200 | | | | |
| 3 | Propylene Glycol | 1000.00 | 50.26 | 1000.00 | 50.26 |
| 4 | Neohesperidin Dihy- | 1.00 | 0.05 | 1.00 | 0.05 |
| | drochalcone | | | | |
| 5 | Peppermint flavor | 3.00 | 0.15 | 3.00 | 0.15 |
| 6 | Glycerol anhydrous | qs to 2 | qs | qs to 2 | qs |
| | | mL | | mL | |
| | Total | 2.00 mL | | 2.00 mL | |

### Manufacturing Process of Example 5a and Example 5b

Examples 5a and 5b were prepared according to the following steps:
i. Apixaban is added in propylene glycol and PEG-200
ii. Heat the mixture at the temperature of 40-45° C. until apixaban is fully dissolved and a
   clear solution forms
iii. Allow the solution to equilibrate at room temperature
iv. Peppermint flavor is added and the volume of the solution is brought to 6 mL
   by adding the required amount of sorbitol solution
v. Pack the solution in amber glass bottles.

| | | Example Sa | | Example Sb | |
|---|---|---|---|---|---|
| | | Apixaban | | Apixaban | |
| | | concentration | | concentration | |
| | | 0.83 mg/mL | | 0.42 mg/mL | |
| No | Ingredients | mg/Dose | % | mg/Dose | % |
| 1 | Apixaban | 5.00 | 0.08 | 2.50 | 0.125 |
| 2 | Propylene Glycol | 4500.00 | 75.00 | 4500.00 | 75.00 |
| 3 | Polyethylene Glycol | 200.00 | 3.33 | 200.00 | 3.33 |
| | 200 | | | | |
| 4 | Peppermint flavor | 3.00 | 0.05 | 3.00 | 0.05 |
| 5 | Sorbitol solution 70% | qs to 6 | qs | qs to 6 | qs |
| | (qs) | mL | | mL | |
| | Total | 6.00 mL | | 6.00 mL | |

### Manufacturing Process of Example 6a and Example 6b

Examples 6a and 6b were prepared according to the following steps:
i. Apixaban is added in propylene glycol and PEG-200
ii. Heat the mixture at the temperature of 40-45° C. until apixaban is fully dissolved and a
   clear solution forms
iii. Allow the solution to equilibrate at room temperature
iv. Peppermint flavor is added and the volume of the solution is brought to 10 mL
   by adding the required amount of sorbitol solution
v. Pack the solution in amber glass bottles.

| | | Example 6a | | Example 6b | |
|---|---|---|---|---|---|
| | | Apixaban | | Apixaban | |
| | | concentration | | concentration | |
| | | 0.5 mg/mL | | 0.25 mg/mL | |
| No | Ingredients | mg/Dose | % | mg/Dose | % |
| 1 | Apixaban | 5.00 | 0.05 | 2.50 | 0.125 |
| 2 | Propylene Glycol | 4500.00 | 45.00 | 4500.00 | 45.00 |
| 3 | Polyethylene Glycol | 200.00 | 2.00 | 200.00 | 2.00 |
| | 200 | | | | |
| 4 | Peppermint flavor | 3.00 | 0.03 | 3.00 | 0.03 |
| 5 | Sorbitol solution 70% | qs to 10 | qs | qs to 10 | qs |
| | (qs) | ml | | ml | |
| | Total | 10.00 mL | | 10.00 mL | |

Concentrated solutions of apixaban are necessary in order to achieve low dosage volumes. The temperature of the mixture of apixaban and optionally co-solvents can be up to 90° C. By dissolving apixaban at temperature up to 90° C. the most concentrated solutions, up to 10 mg/mL, in at least 35% (w/v) of propylene glycol are prepared. The Examples 7-38 were prepared according to the manufacturing process described in the Examples 1-5.

The composition of Examples 7-38 are summarized in Table 1.

TABLE 1
Example 7-38 composition.

| Example | | Concentration | Temp. | |
|---|---|---|---|---|
| No | Formulation | (mg/mL) | (° C.) | Remarks |
| 7 | Apixaban + 35% | 0.25 | 20 | Soluble |
| | Propylene Glycol + 60% | | | |
| | Glycerol anh. | | | |
| 8 | Apixaban + 35% | 0.5 | 20 | Soluble |
| | Propylene Glycol + 60% | | | |
| | Glycerol anh. | | | |
| 9 | Apixaban + 35% | 1.0 | 20 | Partially |
| | Propylene Glycol + 60% | | | soluble |
| | Glycerol anh. | | | |
| 10 | Apixaban + 40% | 1.5 | 20 | Insoluble |
| | Propylene Glycol + 60% | | | |
| | Glycerol anh. | | | |
| 11 | Apixaban + 40% | 2.0 | 20 | Insoluble |
| | Propylene Glycol + 60% | | | |
| | Glycerol anh. | | | |
| 12 | Apixaban + 40% | 1.0 | 35 | Partially |
| | Propylene Glycol + 60% | | | soluble |
| | Glycerol anh. | | | |
| 13 | Apixaban + 40% | 1.0 | 40 | Soluble |
| | Propylene Glycol + 60% | | | |
| | Glycerol anh. | | | |
| 14 | Apixaban + 40% | 1.5 | 45 | Soluble |
| | Propylene Glycol + 60% | | | |
| | Glycerol anh. | | | |
| 15 | Apixaban + 40% | 2.0 | 45 | Soluble |
| | Propylene Glycol + 60% | | | |
| | Glycerol anh. | | | |
| 16 | Apixaban + 100% | 0.25 | 20 | Soluble |
| | Propylene Glycol | | | |
| 17 | Apixaban + 100% | 0.5 | 20 | Soluble |
| | Propylene Glycol | | | |
| 18 | Apixaban + 100% | 1.0 | 20 | Soluble |
| | Propylene Glycol | | | |
| 19 | Apixaban + 100% | 1.85 | 20 | Soluble |
| | Propylene Glycol | | | |
| 20 | Apixaban + 100% | 2.00 | 20 | Partially |
| | Propylene Glycol | | | soluble |
| 21 | Apixaban + 100% | 2.50 | 35 | Soluble |
| | Propylene Glycol | | | |
| 22 | Apixaban + 100% | 3.33 | 35 | Soluble |
| | Propylene Glycol | | | |
| 23 | Apixaban + 100% | 5.00 | 45 | Soluble |
| | Propylene Glycol | | | |
| 24 | Apixaban + 100% | 10.00 | 55 | Soluble |
| | Propylene Glycol | | | |
| 25 | Apixaban + 40% | 0.25 | 20 | Soluble |
| | Propylene Glycol + 3% | | | |
| | PEG-200 + 57% | | | |
| | Glycerol anh. | | | |
| 26 | Apixaban + 40% | 0.5 | 20 | Soluble |
| | Propylene Glycol + 3% | | | |
| | PEG-200 + 57% | | | |
| | Glycerol anh. | | | |
| 27 | Apixaban + 97% | 3.00 | 20 | Soluble |
| | Propylene Glycol + 3% | | | |
| | PEG-200 | | | |
| 28 | Apixaban + 97% | 3.33 | 20 | Partially |
| | Propylene Glycol + 3% | | | soluble |
| | PEG-200 | | | |
| 29 | Apixaban + 97% | 3.33 | 35 | Soluble |
| | Propylene Glycol + 3% | | | |
| | PEG-200 | | | |
| 30 | Apixaban + 97% | 5.00 | 20 | Partially |
| | Propylene Glycol + 3% | | | soluble |
| | PEG-200 | | | |
| 31 | Apixaban + 97% | 5.00 | 35 | Partially |
| | Propylene Glycol + 3% | | | soluble |
| | PEG-200 | | | |
| 32 | Apixaban + 97% | 5.00 | 45 | Soluble |
| | Propylene Glycol + 3% | | | |
| | PEG-200 | | | |
| 33 | Apixaban + 45% | 5.00 | 35 | Partially |
| | Propylene Glycol + 55% | | | soluble |
| | Sorbitol solution | | | |
| 34 | Apixaban + 45% | 5.00 | 45 | Soluble |
| | Propylene Glycol + 55% | | | |
| | Sorbitol solution | | | |
| 35 | Apixaban + 45% | 5.00 | 35 | Partially |
| | Propylene Glycol + 55% | | | soluble |
| | Fructose solution | | | |
| 36 | Apixaban + 45% | 5.00 | 45 | Soluble |
| | Propylene Glycol + 55% | | | |
| | Fructose solution | | | |
| 37 | Apixaban + 45% | 5.00 | 35 | Partially |
| | Propylene Glycol + 55% | | | soluble |
| | Maltitol solution | | | |
| 38 | Apixaban + 45% | 5.00 | 45 | Soluble |
| | Propylene Glycol + 55% | | | |
| | Maltitol solution | | | |

1. A pharmaceutical liquid formulation for oral delivery of apixaban comprising apixaban at a concentration within the range of 0.25 mg/mL to 10 mg/mL and more than 35% (w/v) propylene glycol based on the total formulation, wherein a dose volume of the liquid formulation is equal to or less than 10 mL.

2. The pharmaceutical liquid formulation for oral delivery of apixaban of claim 1 wherein the formulation comprises more than 40% (w/v) propylene glycol based on the total formulation.

3. The pharmaceutical liquid formulation for oral delivery of apixaban of claim 1, wherein the formulation is a solution.

4. The pharmaceutical liquid formulation for oral delivery of apixaban of claim 1, wherein the formulation is essentially non-aqueous.

5. The pharmaceutical liquid formulation for oral delivery of apixaban according to claim 1, further comprising at least one antioxidant.

6. The pharmaceutical liquid formulation for oral delivery of apixaban according to claim 5 wherein the at least one antioxidant is selected from the group consisting of butylated hydroxytoluene (BHT), butylated hydroxyanisole (BHA), propyl gallate, phospholipids, and mixtures thereof.

7. The A pharmaceutical liquid formulation for oral delivery of apixaban according to claim 1 wherein the formulation further comprises at least one co-solvent selected from the group consisting of polyethylene glycol, ethanol, water, glycerol, sorbitol, sucrose, mannitol, maltitol, fructose glucose, and mixtures thereof.

8. The pharmaceutical liquid formulation for oral delivery of apixaban according to claim 1, further comprising one or more sweetening agents and/or flavoring agents.

9. The pharmaceutical liquid formulation for oral delivery of apixaban according to claim 1, further comprising at least one surfactant.

10. A method for preparing a pharmaceutical liquid formulation for oral delivery of apixaban according to claim 1 comprising
i. mixing apixaban and optionally pharmaceutically acceptable excipients in propylene glycol and optionally in co-solvents
ii. applying heat temperature up to 90° C.
iii. maintaining temperature while mixing apixaban until apixaban
dissolves
iv. optionally, bringing to volume with propylene glycol and/or co-solvent and optionally sweetening agents and/or flavoring agents, and
v. optionally, packing in a container

11. A pharmaceutical kit comprising: (i) a container comprising the pharmaceutical liquid formulation according to claim 1, (ii) optionally a calibrated device, and (iii) optionally instructions for administration.

12. The pharmaceutical kit according to claim 11, wherein the container comprising the pharmaceutical liquid formulation is glass and/or plastic material.

13. The pharmaceutical kit according to claim 11, wherein the kit comprises a calibrated device, and the calibrated device is selected from an oral syringe, a dropper, or a spoon.

14. A pharmaceutical liquid formulation for oral delivery of apixaban according to claim 1 for use as a therapy of blood clots following hip or knee replacement, stroke and systemic embolism in people with nonvalvular atrial fibrillation, wherein the dose volume is administered at least partially.

15. The pharmaceutical liquid formulation for oral delivery of apixaban according to claim 14 for use as a therapy of blood clots following hip or knee replacement, stroke and systemic embolism in people with nonvalvular atrial fibrillation, wherein the pharmaceutical liquid formulation for oral delivery of apixaban is administered at least once per day.
